(19) United States
(12) Patent Application Publication
(10) Pub. No.: US 2006/0058865 A1
(43) Pub. Date: Mar. 16, 2006

(54) Delivery system with controlled frictional properties

(76) Inventors: Brian C. Case, Bloomington, IN (US); Jacob A. Flagle, Bloomington, IN (US)

Correspondence Address: DUNLAP, CODDING & ROGERS, P.C., P.O. BOX 16370, OKLAHOMA CITY, OK, 73113, US

(21) Appl. No.: 11/210,998

(22) Filed: Aug. 24, 2005

Related U.S. Application Data
(60) Provisional application No. 60/604,785, filed on Aug. 26, 2004.

(51) Int. Cl. A61F 2/06

(52) U.S. Cl. 623/1.11

(57) ABSTRACT

Delivery systems and methods of making delivery systems are provided. A delivery system according to the invention facilitates delivery of an intraluminal medical device to a point of treatment in a body vessel. A dilator includes a means for resisting relative movement between an associated intraluminal medical device and the dilator during relative movement between the dilator and an associated tubular member disposed about the dilator.

### CROSS-REFERENCE TO RELATED APPLICATION

This application claims priority to U.S. Provisional Application Ser. No. 60/604,785 filed on Aug. 26, 2004, the entire disclosure of which is hereby incorporated herein in its entirety.

### FIELD OF THE INVENTION

The present invention relates to medical devices. More particularly, the invention relates to a delivery system for implantation of an intraluminal medical device in a body vessel.

### BACKGROUND

Minimally invasive techniques and instruments for placement of intraluminal medical devices have been developed over recent years and are frequently used to deliver and deploy an intraluminal medical device at a desired point of treatment. In these techniques, a delivery system is used to carry the intraluminal medical device through a body vessel to the point of treatment. Once the point of treatment is reached, the intraluminal medical device is deployed from the delivery system. The delivery system is subsequently withdrawn from the point of treatment and, ultimately, the body vessel. A wide variety of treatment devices that utilize minimally invasive technology have been developed and include stents, stent grafts, occlusion devices, infusion catheters, prosthetic valves, and the like.

Self-expandable intraluminal medical devices are frequently used in a variety of treatment procedures. For example, self-expandable stents are used to provide support to various vessels and ducts in the cardiovascular and gastrointestinal. systems. Also, prosthetic valves, including prosthetic venous valves, are used to introduce or restore a valving function to a body vessel.

Loading and deployment of the intraluminal medical device involves relative movement between the intraluminal medical device and a sheath or other tubular member housing the device. During loading, the intraluminal medical device typically is held adjacent a dilator. The dilator and intraluminal medical device are then slidingly inserted into a sheath. During deployment, relative movement between the dilator and sheath is used until the intraluminal medical device is fully exposed. Typically, the dilator and intraluminal medical device are caused to slide out of the sheath, either by retraction of the sheath, advancement of the dilator, or a combination of both. Relative movement between the dilator and the intraluminal medical device, however, is typically undesirable as this movement may result in misplacement of the intraluminal medical device relative to a desired point of treatment or other undesirable consequences.

Accordingly, there is a need for delivery systems that permit relative movement between a dilator and sheath and substantially resist relative movement between a dilator and an associated intraluminal medical device during deployment.

### SUMMARY OF EXEMPLARY EMBODIMENTS OF THE INVENTION

The invention provides delivery systems for delivering an intraluminal medical device to a point of treatment in a body vessel. Delivery systems according to the invention have controlled frictional properties that facilitate delivery of the intraluminal medical device included in the delivery system.

In one exemplary embodiment, a delivery system comprises an elongate tubular member having a distal end adapted for insertion into a body vessel. The delivery system also includes a dilator having a distal end adapted for insertion into the body vessel. The dilator is disposed in the tubular member and extends substantially coaxially with the tubular member. The distal end of the dilator has a device chamber formed therein defined by an exterior surface of the dilator. An intraluminal medical device is disposed in the device chamber and radially between the tubular member and the dilator. The exterior surface of the dilator defining the device chamber is formed to resist relative movement between the intraluminal medical device and the dilator during relative movement between the dilator and the tubular member, which occurs during deployment of the intraluminal medical device.

In another exemplary embodiment, a delivery system comprises an elongate tubular member having a distal end adapted for insertion into a body vessel. The delivery system also includes a dilator having a distal end adapted for insertion into the body vessel. The dilator is disposed in the tubular member and extends substantially coaxially with the tubular member. The distal end of the dilator has a device chamber formed therein defined by an exterior surface of the dilator. An intraluminal medical device is disposed in the device chamber and radially between the tubular member and the dilator. The exterior surface of the dilator defining the device chamber is formed to militate against relative movement between the intraluminal medical device and the dilator. At least a portion of the interior surface of the tubular member has lubricious properties to facilitate a sliding of the intraluminal medical device along the interior surface.

The invention also provides methods of producing a delivery system.

An exemplary method comprises the steps of providing a dilator with a device chamber formed by at least a portion of an exterior surface thereof. An intraluminal medical device is provided and disposed in the device chamber of the dilator. A tubular member with at least a portion of an interior surface thereof having lubricious properties is provided. The dilator is inserted into a tubular member to be substantially concentric therewith. The intraluminal medical device is gripped by the device chamber formed by the exterior surface of the dilator and the lubricious interior surface of the tubular member permits the intraluminal medical device to slide thereon.

### BRIEF DESCRIPTION OF THE DRAWINGS

FIG. 1 is a perspective view of a delivery system according to one embodiment of the invention.

FIG. 2 is a sectional view of the distal end of the delivery system illustrated in FIG. 1.

FIG. 3 is a perspective view of the distal end of a dilator of the delivery system illustrated in FIGS. 1 and 2.

FIG. 4 is a perspective view of an alternate embodiment of the dilator illustrated in FIG. 3.

FIG. 5 is a perspective view of an alternate embodiment of the dilator illustrated in FIG. 3.

FIG. 6 is a perspective view of an alternate embodiment of the dilator illustrated in FIG. 3.

FIG. 7 is a sectional view of the distal end of a sheath of the delivery system illustrated in FIGS. 1 and 2.

FIG. 8 is a sectional view of an alternate embodiment of the sheath illustrated in FIG. 7.

FIG. 9 is a flow diagram illustrating a method of producing a delivery system according to the invention.

### DETAILED DESCRIPTION OF EXEMPLARY EMBODIMENTS OF THE INVENTION

The following detailed description and appended drawings describe and illustrate various exemplary embodiments of the invention. The description and drawings serve to enable one skilled in the art to make and use the invention, and are not intended to limit the scope of the invention or its protection in any manner.

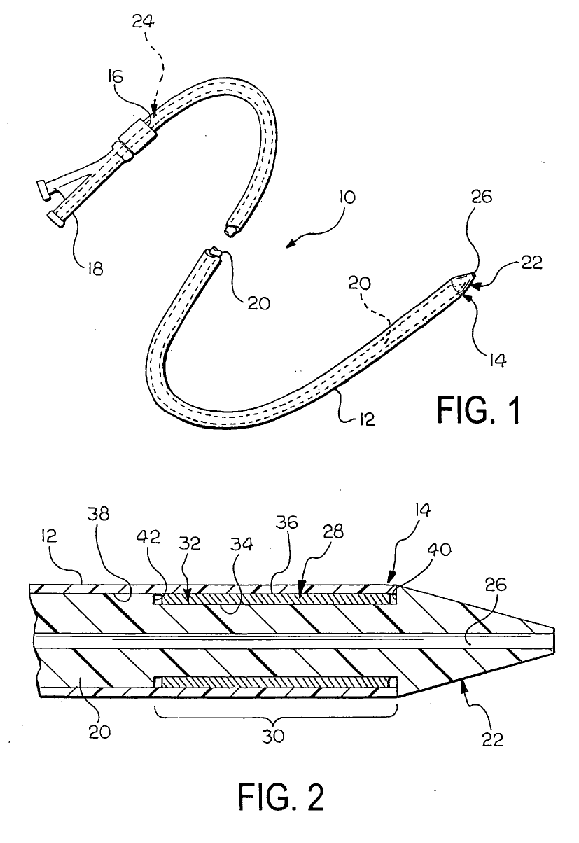

FIGS. 1, 2, and 3 illustrate a delivery system 10 according to one embodiment of the invention. The delivery system 10 includes an elongate sheath or tubular member 12 having a distal end 14 which is insertable in a body vessel and a proximal end 16 that can be coupled to a connector 18 such as a Touhy Borst adapter, for example. The tubular member 12 is formed of a flexible material, such as polyurethane or other suitable polymeric material, for example.

The delivery system 10 includes a dilator 20 disposed within the tubular member 12. As used herein, the term “dilator” refers to an elongate member capable of being disposed within a lumen of a sheath, such as the tubular member 12. The dilator 20 has a tapered distal end 22, which is insertable in the body vessel and a proximal end 24. A lumen 26 is formed by the dilator 20 and extends along the entire length of the dilator 20. The lumen 26 is adapted to receive a wireguide (not shown) or any other suitable member, therein. As used herein, the term “wireguide” refers to elongate members used in minimally invasive procedures to define a path along which other devices can be advanced. The term is considered equivalent in meaning to the term “guidewire” as used in the art. The lumen 26 may aid in guiding the delivery system 10 through the body vessel to a desired point of treatment.

While the embodiment illustrated in FIGS. 1 through 3 includes a lumen 26 that extends along the entire length of the dilator, it is understood that an alternative lumen can be used. For example, a lumen that extends along only a portion of the length of the dilator 26 can be used. Indeed, both over-the-wire and rapid exchange type delivery systems are contemplated and considered to be within the scope of the invention.

FIG. 2 illustrates the distal end of the delivery system 10 illustrated in FIG. 1, including the distal end 14 of the tubular member 12 and the distal end 22 of the dilator 20. An expandable intraluminal medical device 28 is disposed in a device chamber 30 formed in the dilator 20 adjacent to the distal end 22. An exterior surface 32 of the device chamber 30 is adjacent a radially inner portion 34 of the intraluminal medical device 28. A radially outer portion 36 of the intraluminal medical device 28 is adjacent an interior surface 38 of the tubular member 12. The device chamber 30 includes a first annular shoulder 40 formed at a first end thereof and a second annual shoulder 42 formed at a second end thereof.

The intraluminal medical device 28 may be any suitable intraluminal medical device, examples of which include a stent, a prosthetic valve, a filter, an occluder, a distal protection device, a stent graft, and the like. Further, the intraluminal medical device 28 can be a self-expandable device or a device that requires an input of force for expansion, such as a balloon-expandable device. In exemplary embodiments, the intraluminal medical device includes an expandable support frame and a graft member, such as an attached sheet of polymeric or natural material. Examples of such devices include stent grafts and prosthetic valves. Specific examples of suitable self-expandable medial devices for use with delivery systems according to the invention include those described in U.S. Pat. No. 6,200,336 to Pavcnik et al. for a MULTIPLE-SIDED INTRALUMINAL MEDICAL DEVICE; U.S. patent application Ser. No. 10/642,372 of Pavcnik et al. for an IMPLANTABLE VASCULAR DEVICE, filed on Aug. 15, 2003; and U.S. patent application Ser. No. 10/828,716 of Case et al. for an ARTIFICIAL VALVE PROSTHESIS WITH IMPROVED FLOW DYNAMICS, filed on Apr. 21, 2004; the entire disclosures of which are hereby incorporated into this disclosure for the purpose of describing suitable self-expandable medical devices for use with delivery systems according to the invention.

Delivery systems according to the invention are particularly well-suited for use with medical devices that include an expandable support frame and a graft member because the features that resist relative movement between the dilator and intraluminal medical device during deployment are also believed to aid in preventing movement of the graft member prior to expansion of the device, which may be undesirable.

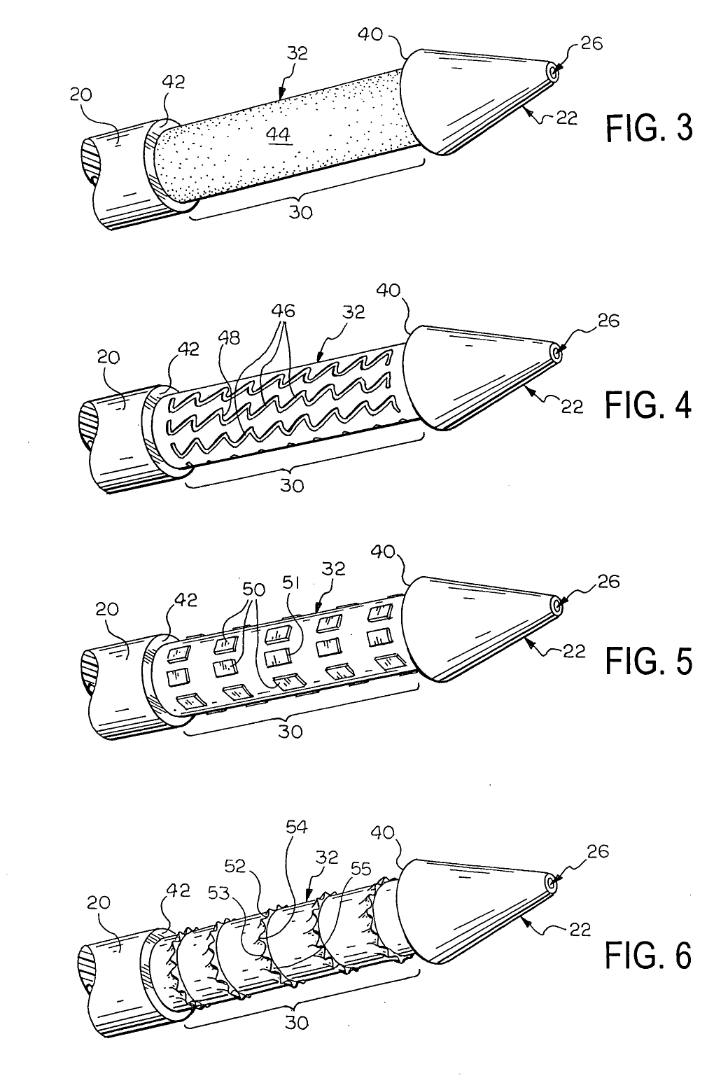

FIG. 3 shows the distal end 22 of the dilator 20 without the tubular member 12 and the intraluminal medical device 28. The exterior surface 32 includes a means for resisting relative movement between the intraluminal medical device 28 and the dilator 20 during relative movement between the dilator 20 and the tubular member 12. Any suitable structure and/or substance can be used as the means for resisting relative movement between the dilator 20 and intraluminal medical device 28. In the embodiment illustrated in FIG. 3, the exterior surface 32 of the dilator 20 in the device chamber 30 includes a coating 44 disposed thereon. The coating 44 resists relative movement between the intraluminal medical device 28 and the exterior surface 32 during loading and deployment of the intraluminal medical device 28 and comprises a suitable means for resisting relative movement between the dilator 20 and the intraluminal medical device 28. The coating 44 has frictional properties, which result in a “gripping” of the intraluminal medical device 28 while the dilator 20 moves relative to the tubular member 12, such as during loading and deployment. Any conventional coating which has the desired frictional properties can be used. Examples of suitable coatings include adhesives, one or more layers of polymeric material, and the like. The coating advantageously provides the desired gripping that resists relative movement between the dilator 20 and the intraluminal medical device 28 but still allows the intraluminal medical device 28 to disassociate from the device chamber 30 of the dilator 20 upon expansion.

Alternatively, the exterior surface 32 of the dilator 20 in the device chamber 30 can be modified to provide the desired resistance to relative movement between the dilator 20 and intraluminal medical device 28. For example, the exterior surface 32 can define a roughened portion. A portion of the exterior surface 32 can be roughened by any suitable technique, such as grit blasting, plasma treatment, and knurling.

FIGS. 4 through 6 illustrate other exemplary embodiments of the invention. Like structure in these Figures has the same reference numerals for clarity. The embodiment illustrated in FIG. 4 includes a plurality of grooves 46 formed in the exterior surface 32 of the dilator 20 at the device chamber 30 as the means for resisting relative movement between the dilator 20 and the intraluminal medical device 28. The grooves 46 have lateral portions 48 formed therewith. The grooves 46 can grip an intraluminal medical device 28 by defining an indentation in which a portion of the device 28 can project.

The embodiment illustrated in FIG. 5 includes a plurality or protuberances 50 formed on the exterior surface 32 of the dilator 20 at the device chamber 30 as the means for resisting relative movement between the dilator 20 and the intraluminal medical device 28. The protuberances 50 can grip an intraluminal medical device 28 by providing a surface 51 that can contact a lateral surface of the device 28, such as the lateral surface of a strut in a support frame.

For illustrative purposes, the protuberances 50 are shown as rectangular in shape. However, protuberances 50 having different shapes can be used. The protuberances 50 can also be provided with a coating, which further militates against relative movement between the radially inner portion 34 of the intraluminal medical device 28, as previously described. The protuberances 50 may be so arranged to permit portions of the intraluminal medical device 28 to be interposed therebetween to resist relative movement between the intraluminal medical device 28 and the exterior surface 32 of the dilator 20. For example, the protuberances 50 can be arranged such that one or more protuberances 50 project into a cell defined by the structure of a support frame of an intraluminal medical device 28, such as a cell defined by a mesh structure of a stent.

The embodiment illustrated in FIG. 6 includes a plurality of annular undulating rings 52 formed on the exterior surface 32 of the dilator 20 of the device chamber 30 as the means for resisting relative movement between the dilator 20 and the intraluminal medical device 28. Each of the plurality of annular undulating rings 52 defines a plurality of peaks 54. One side of each peak 54 has a gradually sloping portion 53 and an abruptly dropping portion 55 that abruptly drops to the exterior surface 32 of the device chamber 30. The gradually sloping portion 53 permits movement of the intraluminal medical device 28 relative to the dilator 20 in one direction while the abruptly dropping portion 55 resists such movement in the opposite direction. As illustrated in FIG. 6, peaks 54 with the gradually sloping portion 53 on a proximal side and the abruptly dropping portion 55 on the distal side provide desirable characteristics.

The inclusion of structural features that permit movement of the intraluminal medical device 28 relative to the dilator 20 in one direction and resist such movement in the opposite direction, such as the peaks 54 illustrated in FIG. 6, may be advantageous for use with intraluminal medical devices in which movement of a component, such as a graft member, that results from movement of the device in one direction is acceptable while movement of the component that results from movement of the device in an opposite direction is undesirable. For example, a device that includes a graft member attached to one end of a support frame but free of the opposite end is expected to benefit from such structural features. An example of such an intraluminal medical device is described in United States Patent Application Publication Number 2003/0191517 to Osborne et al. for an INTRALUMINAL GRAFT ASSEMBLY AND VESSEL REPAIR SYSTEM, the entire disclosure of which is incorporated into this disclosure for the purpose of describing a suitable expandable intraluminal medical device for use in and/or with delivery systems according to the invention.

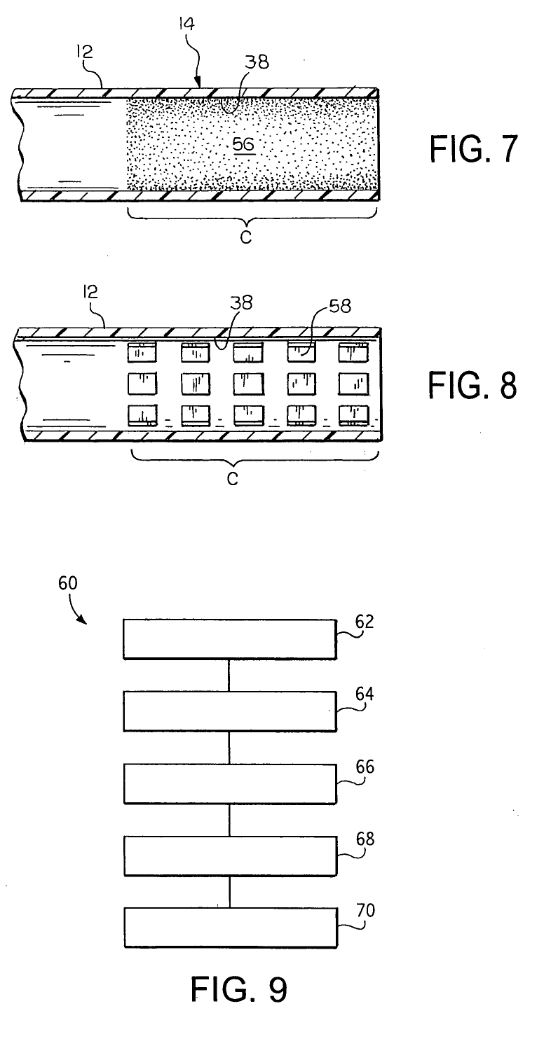

The tubular member 12 can optionally include a means for facilitating relative movement between the intraluminal medical device 28 and the inner surface of the tubular member 12. FIG. 7 illustrates the distal end 14 of the tubular member 12 of the delivery system 10 illustrated in FIGS. 1 and 2 without the dilator 20 and the intraluminal medical device 28. For illustrative purposes, the longitudinal limits of the device chamber 30 of the dilator 20 are represented by the bracket C. The interior surface 38 of the tubular member 12 includes a lubricious coating 56 disposed thereon. The coating 56 facilitates slideable movement of the intraluminal medical device 28 along the interior surface 38 during relative movement between the dilator 20 and the tubular member 12, such as occurs during loading and deployment of the intraluminal medical device 28. In other words, the coating 56 has frictional properties, which result in a “slipping” of the intraluminal medical device 28. Any conventional lubricious coating which has the desired frictional properties can be used. Examples of suitable coatings include silicone, hydrogel polymers, and hydrophilic coatings. Although the coating 56 is shown only on the portion of the interior surface 38 adjacent the device chamber 30, it is understood that a larger portion, indeed even the entire interior surface 38 of the tubular member 12, can be coated without departing from the scope and spirit of the invention. It is also understood that the tubular member 12 could be formed in whole or in part of a lubricious material, such as a polytetrafluoroethylene.

It is understood that other structures and/or compositions can be used to achieve the desired lubricious properties on the interior surface 38 of the tubular member 12. FIG. 8 illustrates another exemplary embodiment of the invention. Like structure in FIG. 8 has the same reference numerals for clarity. The embodiment illustrated in FIG. 8 includes a plurality of protuberances 58 formed on the interior surface 38 of the tubular member 12. This structure reduces the total surface area of the interior surface 38 that contacts an intraluminal medical device disposed within the tubular member 12. As a result, this structure reduces the overall friction between the interior surface 38 and the intraluminal medical device 28.

The protuberances 58 can also be provided with a lubricious coating such as those described herein. Although the protuberances 58 are shown only on the portion of the interior surface 38 adjacent the device chamber 30, it is understood that a large portion, indeed even the entire interior surface 38 of the tubular member 12, can be provided with the protuberances 58. Also, the protuberances 58 can have any suitable size and configuration; the substantially rectangular protuberances 58 illustrated in FIG. 8 are exemplary in nature.

Assembly of the delivery system is facilitated by the invention as herein described, as shown in the flow diagram for a method 60 of production of the delivery system in FIG. 9. The order of the steps in FIG. 9 is exemplary in nature and is not necessary or critical. The dilator 20 is provided with the exterior surface 32 having at least a portion thereon which resists relative movement between the dilator 20 and the intraluminal medical device 28 using a suitable means for resisting such movement, such as the structures and/or method disclosed herein, illustrated by 62. The intraluminal medical device 28 is provided, illustrated by 64, and is disposed around the exterior surface 32 of the dilator 20 in the device chamber 30, illustrated by 66. In an optional step, illustrated by 68, the tubular member 12 is provided with at least a portion of the interior surface 38 having lubricious properties, which can be accomplished by using one of the structures and/or method disclosed herein. The dilator 20 is inserted into the tubular member 12 to be substantially concentric therewith, illustrated by 70. The intraluminal medical device 28 is gripped by the exterior surface 32 of the dilator 20. The lubricious interior surface 38 of the tubular member 12 permits the intraluminal medical device 28 to slide thereon. Thus, the lubricious interior surface 38 of the tubular member 12 and the exterior surface 32 of the dilator 20 cooperate the maintain proper positioning of the intraluminal medical device 28 in the delivery system 10.

In use, the delivery system 10 delivers the intraluminal medical device 28 to a desired location within the body vessel. To deliver the intraluminal medical device 28, a wireguide is placed in the body vessel of the patient by navigating a distal end of the wireguide to or beyond a desired point of treatment. A proximal end of the wireguide is left outside the body of the patient.

When it is desired to insert the delivery system 10 in the body vessel, the proximal end of the wireguide is inserted into the lumen 26 of the dilator 20 at the distal end 22. The distal end 22 of the dilator 20 is caused to enter the body vessel along the wireguide and to be moved to the desired point of treatment. Deployment of the intraluminal medical device 28 at a desired point of treatment can be accomplished by causing the intraluminal medical device 28 and the distal end 22 of the dilator 20 to be slidingly moved out of the tubular member 12, either by retracting the tubular member 12 or advancing the dilator 20. The lubricious interior surface 38 of the tubular member 12 permits the intraluminal medical device 28 to slide adjacent thereto. The exterior surface 32 having at least a portion thereof which resists relative movement between the dilator 20 and the intraluminal medical device 28 operates to substantially hold the intraluminal medical device 28 in place during relative movement between the dilator 20 and the tubular member 12, thus facilitating deployment and resisting undesirable movement of the intraluminal medial device 28 relative to the dilator 20. Thus, the intraluminal medical device 28 is permitted to slide relative to the tubular member 12 and movement of the intraluminal medical device 28 relative to the dilator 20 is resisted. Additionally, the force exerted on the intraluminal medical device 28 by the dilator 20 is dispersed over a larger surface area of the intraluminal medical device 28 compared to prior art structures and methods which concentrate such force on the ends of the intraluminal medical device 28.

A desired result is that the interior surface 38 of the tubular member 12 is more lubricious than the exterior surface 32 of the dilator 20. This facilitates the intraluminal medical device 28 being held relative to the dilator 20 and sliding relative to the tubular member 12. Stated differently, the coefficient of friction of the interior surface 38 can be less than coefficient of friction of the exterior surface 32.

From the foregoing description, one ordinarily skilled in the art can easily ascertain the essential characteristics of this invention and, without departing from the spirit and scope thereof, can make various changes and modifications to the invention to adapt it to various usages and conditions. For example, although the invention is described and illustrated in the context of an over-the-wire delivery system, one of ordinarily skill in the art can adapt a rapid exchange delivery system in accordance with the invention without departing from the spirit and scope of the invention.

What is claimed is:

1. An intraluminal medical device delivery system, comprising:
an elongate tubular member having a distal end adapted for insertion into a body vessel;
a dilator having a distal end adapted for insertion into the body vessel, the dilator disposed in the tubular member and extending substantially coaxially with the tubular member, the distal end of the dilator having a device chamber formed therein defined by an exterior surface of the dilator;
an intraluminal medical device disposed radially between the tubular member and the dilator in the device chamber; and
a means for resisting relative movement between the intraluminal medical device and the dilator during relative movement between the dilator and the tubular member.

2. The delivery system according to claim 1, wherein the means for resisting relative movement between the intraluminal medical device and the dilator during relative movement between the dilator and the tubular member comprises a coating disposed on the exterior surface in the device chamber.

3. The delivery system according to claim 1, wherein the means for resisting relative movement between the intraluminal medical device and the dilator during relative movement between the dilator and the tubular member comprises one or more projections defined by the exterior surface in the device chamber.

4. The delivery system according to claim 3, wherein the one or more projections comprise annular rings.

5. The delivery system according to claim 4, wherein each annular ring defines a plurality of peaks.

6. The delivery system according to claim 5, wherein each peak has a gradually sloping portion and an abruptly dropping portion.

7. The delivery system according to claim 6, wherein the abruptly dropping portion is positioned distal to the gradually sloping portion.

8. The delivery system according to claim 1, wherein the means for resisting relative movement between the intraluminal medical device and the dilator during relative movement between the dilator and the tubular member comprises one or more grooves defined by the exterior surface in the device chamber.

9. The delivery system according to claim 1, wherein the tubular member includes a means for facilitating relative movement between the intraluminal medical device and the tubular member.

10. The delivery system according to claim 9, wherein the means for facilitating relative movement between the intraluminal medical device and the tubular member comprises a coating disposed on an inner surface of the tubular member.

11. The delivery system according to claim 9, wherein the means for facilitating relative movement between the intraluminal medical device and the tubular member comprises one or more inwardly projecting protuberances.

12. The delivery system according to claim 1, wherein the intraluminal medical device comprises a self-expandable intraluminal medical device.

13. The delivery system according to claim 1, wherein the intraluminal medical device comprises a prosthetic valve.

14. The delivery system according to claim 1, wherein the intraluminal medical device comprises a support frame and a graft member.

15. An intraluminal medical device delivery system, comprising:
a dilator defining a device chamber;
a coating disposed in the device chamber;
an intraluminal medical device disposed in the device chamber adjacent the coating; and
an elongate tubular member disposed about the dilator and adjacent the intraluminal medical device.

16. The delivery system according to claim 15, wherein the tubular member includes a means for facilitating relative movement between the intraluminal medical device and the tubular member.

17. The delivery system according to claim 16, wherein the means for facilitating relative movement between the intraluminal medical device and the tubular member comprises a coating disposed on an inner surface of the tubular member.

18. The delivery system according to claim 16, wherein the means for facilitating relative movement between the intraluminal medical device and the tubular member comprises one or more inwardly projecting protuberances.

19. The delivery system according to claim 15, wherein the intraluminal medical device comprises a self-expandable intraluminal medical device.

20. An intraluminal medical device delivery system, comprising:
a dilator defining a device chamber;
a first coating disposed in the device chamber;
an intraluminal medical device disposed in the device chamber adjacent the first coating;
an elongate tubular member disposed about the dilator and adjacent the intraluminal medical device;
a second coating disposed on the tubular member and adjacent the intraluminal medical device;
wherein the first coating resists relative movement between the dilator and the intraluminal medical device during relative movement between the dilator and the tubular member.
